(19) United States
(12) Patent Application Publication
(10) Pub. No.: US 2014/0004157 A1
(43) Pub. Date: Jan. 2, 2014

(54) AGENT FOR DELAYING DIGESTION AND ABSORPTION OF LIPID

(71) Applicant: FUJI OIL COMPANY LIMITED, OSAKA (JP)

(72) Inventors: Toshiharu ARISHIMA, Osaka (JP); Makiko KOJIMA, Ibaraki (JP)

(21) Appl. No.: 13/927,407

(22) Filed: Jun. 26, 2013

(30) Foreign Application Priority Data
Jun. 27, 2012 (JP) 2012-143835

(51) Int. Cl. A61K 31/215; A61K 9/107

(52) U.S. Cl. CPC A61K 31/215; A61K 9/107
USPC 424/400; 514/547

(57) ABSTRACT

An object is to achieve a big effect about an agent for delaying digestion and absorption of lipid or a lipase inhibitor which is composed of specific triacylglycerol and fat-soluble and which exerts the effect more mildly with small used amount. An oil-in-water emulsion having 2 μm or less of average particle diameter, in which a melting point of a fat composition which composes an oil phase of the emulsion is 37° C. or less and the fat composition includes 30% by weight or more of BX2 triacylglycerol (B represents Behenic acid and X represents fatty acid having 4 to 24 carbon atoms) is simultaneously ingested with ingesting fat-containing food. An amount of the BX2 triacylglycerol with respect to fat amount derived from fat-containing food ingested simultaneously is preferably 0.5 to 20% by weight.

### TECHNICAL FIELD

The present invention relates to an agent for delaying digestion and absorption of lipid, including oil-in-water emulsion having specific fat composition and particle size as an active ingredient, and a method for reinforcing lipase inhibitor including specific triacylglycerol as an active ingredient.

### BACKGROUND ART

In recent years, obesity population increases because of an excessive intake of fat from meals and lack of physical activity. Obesity is main factor of an arteriosclerosis disease that causes diabetic, hyperlipemia, and hypertension etc., and the prevention is socially paid to attention. Although obesity can be prevented by consuming energy by exercise, the time spent in the exercise is restricted in the lifestyle in recent years, and therefore, energy consumption is not enough.

Up to now, some studies for amount and quality of lipid ingested from food have been performed. In addition to a simply reduction of fat content in food, low absorptive fat utilizing the fact that an absorption of an O/W emulsion having particle diameter of 2 μm or less is slow (Patent Document 1), making low calorie fat utilizing the fact that behenic acid generated by hydrolysis has low digestion and absorption (Patent Document 2), and substitution for fat alternative (Patent Document 3) etc. have been considered. In almost every case, however, palatability of traditional food is lost by reducing an amount of fat, or by being limited a method of ingesting fat to ingestion of food containing emulsion, or by changing liquid fat to high-melting point fat composition. Thus, a development of a material to be able to contribute to an obesity control while maintaining an amount of fat and kinds of fat as used in traditional food has been desired.

On the other hand, if digestion with lipase can be inhibited or delayed to certain level, transportation of the neutral fat in the blood is delayed even if absorbed, and the accumulated amount of fat decreases because it is late from the rising timing of LPL activity with insulin, it is thought to lead to the anti-obesity. A water-soluble lipase inhibitor being included in plant material or an extract thereof is proposed in Patent Documents 4 and 5. However, these extracts have not come on the market because these extracts have poor compatibility with fat, a method for administering the water-soluble inhibitor is limited to separately take with beverages etc. at meal time and is troublesome, or effect of the extracts is insufficient.

A use of tetrahydrolipstatin as a gastrointestinal lipase inhibitor, as a fat-soluble substance capable of dissolving in fat, is proposed in Patent Document 6. The inhibitor is said to directly covalently bind with lipase itself to inactivate it. The effect of the inhibitor is considerably strong and in some cases, results in diarrheal symptom. Thus, there remains concern about safety when the inhibitor is used for food. Therefore, there is a need for a fat-soluble lipase inhibitor that exerts the effect more mildly. As a fat-soluble lipase inhibitor that exerts the effect more mildly, Patent Document 7 describes a method for inhibiting lipid absorption mildly by substituting a part of conventional fat with triacylglycerol composed of long-chain saturated fatty acid and short-chain fatty acid. However, further reduction of amount of lipase inhibitor is desired.

### PRIOR ART DOCUMENTS

### Patent Documents

Patent Document 1: JP 2012-70653 A
Patent Document 2: JP H01-085040 A
Patent Document 3: JP S64-34244 A
Patent Document 4: JP 2002-179586 A
Patent Document 5: JP 2002-275077 A
Patent Document 6: U.S. Pat. No. 4,598,089
Patent Document 7: JP 2006-151875 A

### Non-Patent Documents

Non-Patent Document 1: Am J Physiol 271: G172-183 1996
Non-Patent Document 2: Am J Clin Nutr 1999; 70: 1096-1106

### SUMMARY OF THE INVENTION

### Problems to be Solved by the Invention

An object of the present invention is to achieve a big effect about an agent for delaying digestion and absorption of lipid or a lipase inhibitor which is composed of specific triacylglycerol and fat-soluble and which exerts the effect more mildly with small used amount.

### Means for Solving the Problems

For determining a lipase activity inhibition of the agent for delaying digestion and absorption of lipid or the lipase inhibitor described in Patent Document 7 after pre-blending with other fat, about 5% by weight of used amount is necessary. Further, for achieving similar effect in a short term in vivo, it is necessary to increase the amount.

The present inventors have intensively studied for determination of a condition for obtaining lipase inhibition effect with small used amount. As a result, the present inventors have found that the delaying effect or the lipase activity inhibition effect of the agent for delaying digestion and absorption of lipid or the lipase inhibitor can be enhanced with used amount smaller than in the past not by pre-blending the agent or the inhibitor directly with fat, but by forming oil-in-water emulsion having 2 μm or less of average particle diameter, which is smaller than 10-100 μm of particle diameter of food-derived fat emulsion in inside of the body (non-Patent Documents 1 and 2). The present invention has been completed on the basis of these findings.

That is, the present invention relates to:

(1) An agent for delaying digestion and absorption of lipid, comprising an oil-in-water emulsion having 2 μm or less of average particle diameter as an active substance, wherein a melting point of a fat composition which composes an oil phase of the emulsion is 37° C. or less, and wherein the fat composition comprises 30% by weight or more of BX2 triacylglycerol wherein B represents Behenic acid and X represents fatty acid having 4 to 24 carbon atoms;

(2) The agent for delaying digestion and absorption of lipid according to (1), wherein an amount of the BX2 triacylglycerol with respect to fat amount derived from fat-containing food ingested simultaneously with the agent is 0.5 to 20% by weight;

(3) The agent for delaying digestion and absorption of lipid according to (1), wherein the oil-in-water emulsion is used in dried form;

(4) An anti-obesity agent comprising the agent for delaying digestion and absorption of lipid according to (1);

(5) A method for enhancing a lipase inhibitor, comprising preparing an oil-in-water emulsion having 2 μm or less of average particle diameter and administering the oil-in-water emulsion, wherein a melting point of a fat composition which composes an oil phase of the emulsion is 37° C. or less, and wherein the fat composition comprises 30% by weight or more of BX2 triacylglycerol wherein B represents Behenic acid and X represents fatty acid having 4 to 24 carbon atoms;

(6) The method for enhancing a lipase inhibitor according to (5), wherein an amount of the BX2 triacylglycerol with respect to fat amount derived from fat-containing food ingested simultaneously with the oil-in-water emulsion is 0.5 to 20% by weight.

### Effect of the Invention

By using oil-in-water emulsion of the present invention, the BX2 triacylglycerol can strongly inhibit hydrolysis of another fat ingested simultaneously and can delay a digestion and absorption speed in spite of small used amount of 0.5-20% by weight, and thereby, can effectively act to prevention and treatment of an obesity by an excess ingestion of lipid and a disease that occurs due to obesity.

### BRIEF DESCRIPTION OF DRAWINGS

FIG. 1 The figure shows time course of each serum neutral lipid level after loading emulsion C of the present invention and control emulsion D in human study of Example 6.

FIG. 2 The figure shows Area Under Curve (AUC), calculated from a graph of FIG. 1 showing a time course of each serum neutral lipid level.

### MODE FOR CARRYING OUT THE INVENTION

### (Fat Composition)

For the oil-in-water emulsion of the present invention in which a melting point of a fat composition which composes an oil phase of the emulsion is 37° C. or less and the fat composition includes 30% by weight or more of BX2 triacylglycerol (B represents Behenic acid and X represents fatty acid having 4 to 24 carbon atoms), the fat composition includes 30% by weight or more of BX2 triacylglycerol having one or more of behenic acid, such as BOO (in which behenic acid is at the 1-position and oleic acid is at the 2,3-positions, melting point: 31° C.), OBO (in which behenic acid is at the 2-position and oleic acid is at the 1,3-positions, melting point: 25° C.), BLiLi (in which behenic acid is at the 1-position and linoleic acid is at the 2,3-positions, melting point: 13° C.), BLnLn (in which behenic acid is at the 1-position and linolenic acid is at the 2,3-positions, melting point: 9° C.) and BClCl (in which behenic acid is at the 1-position and caprylic acid is at the 2,3-positions, melting point: 34° C.)

Alternatively, fat composition including 30% by weight or more of BX2 triacylglycerol having melting point of more than 37° C. which is higher than temperature of human, such as BBuBu (in which behenic acid is at the 1-position and butyric acid is at the 2,3-positions, melting point: 38° C.) and BOB (in which oleic acid is at the 2-position and behenic acid is at the 1,3-positions, melting point: 54° C.), can also be used on the condition that the fat composition has 37° C. or less of melting point by including other fat having lower melting point.

BX2 triacylglycerol having 37° C. or less of melting point is preferable. Among them, BOO (in which behenic acid is at the 1-position and oleic acid is at the 2,3-positions) and OBO (in which behenic acid is at the 2-position and oleic acid is at the 1,3-positions) are more preferable.

The BX2 triacylglycerol is generally obtained by mixing a fat containing behenic acid derived from an animal, a plant or a fish or an ester thereof with animal and/or plant fat including other fat or an ester thereof in appropriate proportion, and then subjecting the mixture to interesterification with a known enzymatic method using an enzyme such as lipase or a non-enzymatic method using an alkali catalyst such as sodium methylate. Alternatively, the BX2 triacylglycerol is also synthesized by mixing behenic acid and other fat and glycerol and then subjecting the mixture to an enzymatic or non-enzymatic process. Examples of a plant fat include rapeseed oil, palm oil, cottonseed oil, sunflower oil, corn oil, canola oil and the like. Examples of an animal fat include beef tallow, lard, fish oil and the like. The triacylglycerol thus obtained can be then subjected to conventional processing treatment such as distillation and fractionation to be concentrated.

It is necessary to include 30% by weight or more of the BX2 triacylglycerol in the fat composition. When the amount is less than 30% by weight, it is difficult to obtain the expected effect and it is necessary to increase a used amount of the emulsion of the present invention with respect to fat amount derived from fat-containing food ingested simultaneously. It is preferable that the amount is more than 50% by weight. When the amount is too high, melting point may be high depending on the formulation.

As previously mentioned, it is important that melting point of the fat composition is 37° C. or less. The fat composition of the present invention should be molten to act on lipase when the triacylglycerol which composes the fat composition functions as a lipase inhibitor. For applying the agent of the present invention for delaying digestion and absorption of lipid to human body or isothermal animal, the agent should be molten at 37° C. which is body temperature of human or isothermal animal.

### (Oil-in-Water Emulsion)

It is necessary to adjust the oil-in-water emulsion composed of the above mentioned fat composition to 2 μm or less, preferably 1 μm or less of median diameter for effectively inhibiting or delaying hydrolysis by pancreatic lipase to lipid ingested simultaneously. The measurement of the median diameter can be performed with a conventional laser granulometry meter as a median diameter of the volume standard.

For production of fine particle of oil-in-water emulsion, emulsifier or protein having emulsification property which is generally used for food can be used. Examples thereof include glycerin fatty acid ester, sucrose fatty acid ester, Polyglycerol fatty acid ester, sorbitan fatty acid ester, skim milk powder, whole milk powder and casein etc. In addition, a method for emulsification is not particularly limited, and general emulsification equipment can be used. Examples thereof include high-pressure homogenizer, homomixer, ultrasonic oscillator and “Nanomizer” (NANOMIZER Inc.) etc.

### (Used Amount)

As for an used amount of the oil-in-water emulsion of the present invention, it is preferable that an amount of the BX2 triacylglycerol derived from the oil-in-water emulsion is 0.5 to 20% by weight, preferably 1 to 10% by weight, more preferably 3 to 8% by weight with respect to total fat amount derived from fat-containing food ingested simultaneously. When the used amount is too small, an effect delaying digestion and absorption of lipid and an effect of lipase inhibition become weak and it is not preferable. On the other hand, even when the used amount exceeds the upper limit, the effect delaying digestion and absorption of lipid and the effect of lipase inhibition exceeding the upper limit cannot be obtained. Therefore, it is not necessary to use it exceeding the upper limit.

The oil-in-water emulsion of the present invention can be administered to a subject such as human or isothermal animal in various ways. Generally, when fat-containing food is ingested, the oil-in-water emulsion of the present invention can be ingested simultaneously. By adding the oil-in-water emulsion of the present invention to beverage etc., it can be ingested simultaneously with fat-containing food, for example, margarine, fried food, confectioneries such as chocolate, bakery, processed meat products such as ham and sausage, processed marine products such as kamaboko (boiled fish paste) and chikuwa (a tube-shaped fish paste cake). Alternatively, a powder obtained by drying the oil-in-water emulsion can be added to the above foods in advance.

### EXAMPLES

Hereinafter, the present invention will be explained in more detail by reference to the following Examples. In Examples, both of % and part are based on weight.

### Production Example

### Preparation of BX2 Triacylglycerol

Fifty parts of a high-oleic sunflower oil having an iodine value of 84 and 50 parts of ethyl behenate having a purity of 95% were mixed and then subjected to interesterification using a 1,3-position specific lipase (Novozaymes, Lipozyme RM-IM) to obtain a reaction oil. The reaction oil was distilled to remove esters and then 100 parts of hexane was added followed by subjecting to crystallization and fractionation at low temperature to obtain 30 parts of BOO fraction (BX2 triacylglycerol content: 68% by weight, melting point: 28° C.) containing BOO as a major component.

### Example 1

### Inhibition of Hydrolysis of Emulsion in Cow Milk by BX2

To 25% by weight of the obtained BOO fraction, 1.0 part by weight of sucrose fatty acid ester (S-570, manufactured by Mitsubishi-Kagaku Foods Corporation), 0.5 part by weight of lecithin (Sunlecithin S), 6 parts by weight of casein sodium, 0.6 part by weight of phosphate (sodium polyphosphate), 66 parts by weight of water and 0.8 part by weight of sugar were added, and then emulsifying the mixture with ultrasonic oscillator for 1 minute to obtain an emulsion having 0.9 μm of median diameter. The emulsion was diluted 27-fold in distilled water to obtain refined BOO emulsion (emulsion A, fat content: 0.9% by weight). The measurement of the median diameter was performed with SALD-2000A manufactured by Shimadzu Corporation.

Commercially available non-homogenized milk (Tomo dairy Cooperatives pasteurized milk, milk fat: 3.5%, particle diameter: 3.3 μm) was diluted 4-fold in distilled water. To 300 μl of the dilution, 1% by weight, 3% by weight or 5% by weight of the above refined BOO emulsion was added (As BX2 triacylglycerol with respect to whole fat, 0.68, 2.0 or 3.4% by weight) to prepare substrate. To the substrate, 100 units of pig pancreatic lipase (Sigma Co.) was added and reacted at 37° C. for 30 minutes, and then adding 3 ml of extraction solvent (mixture of chloroform/heptane/methanol=49 parts/49 parts/2 parts) and stirring well. The resultant mixture was subjected to centrifugation at 2,500 rpm for 5 minutes, and then removing an upper layer. To the under layer, 1 ml of copper reagent (prepared by dissolving 2.98 g of triethanolamine, 2.42 g of copper nitrate, and 0.48 g of NaOH into 200 ml of water and then adding 66 g of NaCl) was added and stirred for 10 minutes. The resultant mixture was subjected to centrifugation at 2,500 rpm for 10 minutes, and 1.5 ml of the upper layer was recovered. To the upper layer, 1.5 ml of coloring reagent (prepared by dissolving 0.2 g of bathocuproine and 0.1 g of butylhydroxyanisol into 200 ml of chloroform) was added followed by determining quantity of free fatty acid by absorbance at OD480. Relative activity with respect to hydrolysis degree of the dilution milk was shown in table 1. As shown in table 1, it was found that hydrolysis was delayed about 20% in the system of adding 1% by weight, about 28% in the system of adding 3% by weight, and about 30% in the system of adding 5% by weight, of refined BOO emulsion as fat with respect to total fat.

### Comparative Example 1

### Inhibition of Hydrolysis of Emulsion in Cow Milk by Non-BX2 Fat 1

By using soybean oil instead of the BOO fraction, 1% by weight, 3% by weight or 5% by weight of refined emulsion (oil content: 0.9% by weight) having 0.9 μm of median diameter obtained by using soybean oil was added to the dilution of the commercially available non-homogenized milk, and hydrolysis degree was determined in the same manner as Example 1. The obtained hydrolysis degree was shown in table 1. Even when additive amount was increased, significant delaying effect of refined emulsion of soybean oil was not observed.

### Comparative Example 2

### Inhibition of Hydrolysis of Emulsion in Cow Milk by Non-BX2 Fat 2

By using a refined emulsion prepared by subjecting commercially available coffee creamer (Nestle krematop, median diameter: 0.6 μm) to 28-fold dilution in distilled water instead of the refined emulsion obtained from BOO fraction in Example 1, 1% by weight, 3% by weight or 5% by weight of the refined emulsion (oil content: 0.9% by weight) was added to the dilution of the commercially available non-homogenized milk, and hydrolysis degree was determined in the same manner as Example 1. The obtained hydrolysis degree was shown in table 1. Even when additive amount was increased, significant delaying effect of commercially available coffee creamer was not observed.

TABLE 1
Relative value of hydrolysis of cow milk when
adding refined emulsion derived from different kind of fat

| | Substrate fat/ | Substrate fat/ | Relative |
|---|---|---|---|
| Test | emulsion fat | BX2 | activity (%) |
| Control (cow milk) | 100/0 | 100/0 | 100 |
| Example 1 | 100/1 | 100/0.68 | 80 |
| (Cow milk/BOO fraction | 100/3 | 100/2.0 | 72 |
| emulsion) | 100/5 | 100/3.4 | 62 |
| Comparative Example 1 | 100/1 | 100/0 | 95 |
| (Cow milk/soybean oil | 100/3 | 100/0 | 97 |
| emulsion) | 100/5 | 100/0 | 94 |
| Comparative Example 2 | 100/1 | 100/0 | 98 |
| (Cow milk/commercially | 100/3 | 100/0 | 115 |
| available coffee creamer) | 100/5 | 100/0 | 115 |

### Example 2

### Inhibition of Hydrolysis of Soybean Oil Emulsion by BX2

Instead of commercially available cow milk in Example 1, emulsion B (oil content: 0.9% by weight) prepared by adding 1.0 part by weight of sucrose fatty acid ester, 0.5 part by weight of lecithin, 6 parts by weight of casein sodium, 0.6 part by weight of phosphate, 66 parts by weight of water and 0.8 part by weight of sugar to 25 parts by weight of soybean oil followed by emulsifying the mixture with ultrasonic oscillator for 1 minute to obtain an emulsion having 2.8 μm of median diameter and then subjecting the emulsion to 28-fold dilution in distilled water was used. To the emulsion B, 1% by weight, 3% by weight or 5% by weight of the refined BOO emulsion used in Example 1 was added and hydrolysis degree was determined in the same manner as Example 1. The result is shown in table 2. It was found that hydrolysis was delayed about 30% in the system of adding 0.68% by weight of BX2 triacylglycerol with respect to whole fat, and delayed about 50% in the system of adding 3.4% by weight.

### Comparative Example 3

### Inhibition of Hydrolysis of Emulsion Obtained by Emulsification after Mixing

As for the emulsion formulation shown in Example 2, instead of the soybean oil, fat in which soybean oil/BOO fraction were pre-blended at the weight ratio of 100/1, 100/3, 100/5 or 100/10 was prepared. An emulsion having 2.8 μm of median diameter was prepared by using the pre-blended fat in the same formulation of Example 2. Lipase was added to the emulsion and hydrolysis degree was determined in the same manner as Example 1. As shown in table 2, the emulsion obtained by pre-blending BX2 triacylglycerol into soybean oil could not show sufficient effect of delaying hydrolysis in small amount. It was shown that about 10 times amount of BX2 triacylglycerol compared to Example 2 was necessary.

TABLE 2
Relative value of hydrolysis of soybean oil
emulsion due to a difference of addition method

| | Substrate fat/ | Substrate | Relative |
|---|---|---|---|
| Test | emulsion fat | fat/BX2 | activity (%) |
| Soybean oil emulsion (2.8 μm) | 100/0 | 100 | 100 |
| Example 2 | 100/1 | 100/0.68 | 70 |
| Soybean oil emulsion/BOO | 100/3 | 100/2.0 | 49 |
| fraction emulsion | 100/5 | 100/3.4 | 47 |
| | 100/10 | 100/6.8 | 45 |
| Comparative Example 3 | 100/1 | 100/0.68 | 101 |
| (Soybean oil/BOO fraction) | 100/3 | 100/2.0 | 101 |
| emulsion | 100/5 | 100/3.4 | 91 |
| | 100/10 | 100/6.8 | 75 |

### Examples 3-5

As for the refined emulsion of BOO fraction in Example 1, the refined emulsion with same formulation but having different median diameter, 0.5 μm, 0.9 μm or 1.5 μm was prepared. In the same manner as Example 1, 1% by weight, 3% by weight or 5% by weight of the refined emulsion was added to the dilution of the commercially available non-homogenized milk and hydrolysis degree was determined. Result was shown in table 3. The delaying effect was observed in any particle diameters. It was confirmed that the emulsion having 0.9 μm diameter tended to show greater effect of delaying.

TABLE 3

| | Substrate fat/ | Substrate fat/ | Relative |
|---|---|---|---|
| Test | emulsion fat | BX2 | activity (%) |
| Control (cow milk) | 100/0 | 100/0 | 100 |
| Example 3 | 100/1 | 100/0.68 | 85 |
| (Cow milk/0.5 μm BOO | 100/3 | 100/2.0 | 77 |
| fraction emulsion) | 100/5 | 100/3.4 | 66 |
| Example 4 | 100/1 | 100/0.68 | 81 |
| (Cow milk/0.9 μm BOO | 100/3 | 100/2.0 | 69 |
| fraction emulsion) | 100/5 | 100/3.4 | 65 |
| Example 5 | 100/1 | 100/0.68 | 88 |
| (Cow milk/1.5 μm BOO | 100/3 | 100/2.0 | 76 |
| fraction emulsion) | 100/5 | 100/3.4 | 69 |

### Example 6

### Preparation of Beverage for Human Study

To 1.3 g of BOO fraction obtained in Production Example, 0.03 g of sucrose fatty acid ester (S-1670, manufactured by Mitsubishi-Kagaku Foods Corporation), 0.02 g of lecithin (Sunlecithin S), 0.01 g of sorbitan fatty acid ester (Poem S-60V, manufactured by Riken Vitamin Co., Ltd.), 0.007 g of phosphate (sodium hexametaphosphate), 5.0 g of water, and then pre-emulsified with a homomixer. The pre-emulsified mixture was applied to homogenizer (econaizer LABO-01, manufactured by SANMARU MACHINERY Co., LTD.) to obtain emulsion C having 0.9 μm of median diameter. In the same manner as above except for using coconut oil instead of BOO fraction, emulsion D was obtained.

To mixed solution including 1.65 g of instant coffee (Blendy, manufactured by AGF), 9.75 g of granulated sugar and 177.1 g of water, 6.5 g of emulsion C or emulsion D was added, and 0.98 g of sucrose fatty acid ester (S-1670, manufactured by Mitsubishi-Kagaku Foods Corporation) was further added, and then pH was adjusted to pH 6.5 with sodium bicarbonate to obtain 196 g of coffee beverage as a beverage for human study. The obtained coffee beverage was applied for human study after filling into can and then sterilized with retort sterilizer. Coffee beverage made from emulsion C is a beverage for test beverage consumption group, and coffee beverage made from emulsion D is a beverage for control beverage consumption group.

### (Procedure of Human Study)

This Example was performed with the double blind crossover method. The subjects for the study were men and women who were 20 years old or more and less than 70 years old volunteers, and whose serum neutral fat (serum triacylglycerol) value when being hungry was about 150-250 mg/dl. Meal for intake was a can (196 g) of the coffee beverage, a slice of bread (Hotel morning 6 slices, manufactured by First baking Co., Ltd.) and 14 g of margarine (Rama butter taste, manufactured by J-OIL MILLS, INC.). A ratio of the BX2 triacylglycerol in a total lipid of the taken meal was 5.4%. Blood sample was corrected every hour from just before ingesting the meal to 6 hours after the ingestion, and change of the serum neutral fat value was examined.

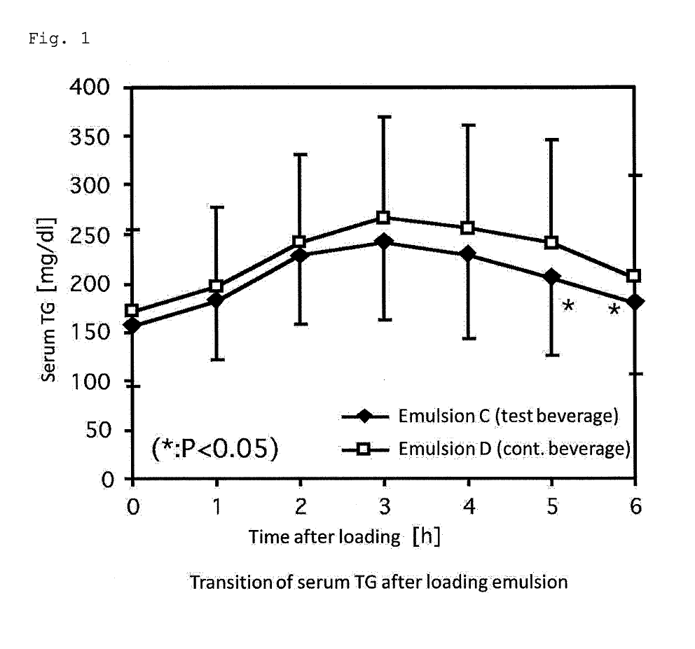

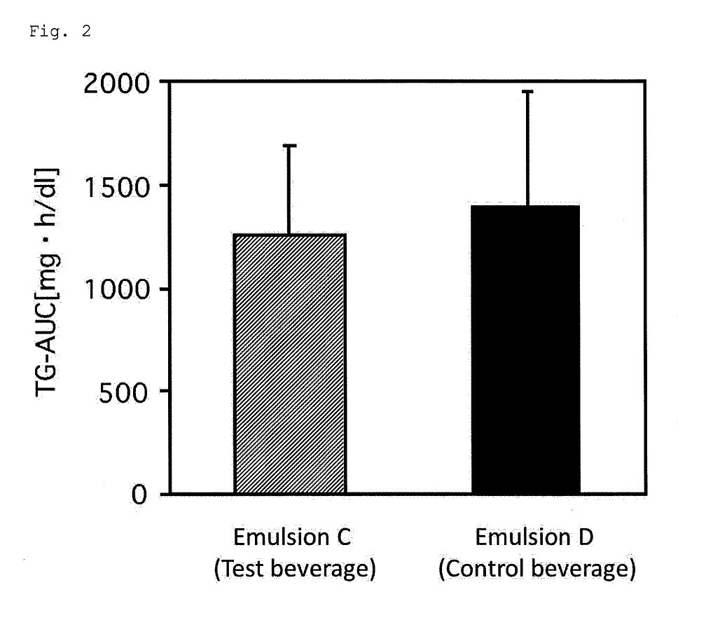

Transition of the serum neutral fat value is shown in FIG. 1. The rise of the serum neutral fat value could be significantly reduced at 5 hours and 6 hours after ingesting the meal in test beverage consumption group compared to control beverage consumption group. In addition, from the Area Under Curve of serum neutral fat value after ingesting the meal (FIG. 2), it was reduced 12.3% in test beverage consumption group compared to control beverage consumption group. Thus, it was confirmed that the BX2 triacylglycerol-containing refined emulsion was effective for reducing the rise of the serum neutral fat value due to delaying absorption of fat.

### INDUSTRIAL APPLICABILITY

An obesity by an excess ingestion of lipid and a disease that occurs due to obesity can be prevented by simultaneously ingesting the BX2 triacylglycerol-containing refined emulsion with ingesting fat-containing food.

1. An agent for delaying digestion and absorption of lipid, comprising an oil-in-water emulsion having 2 μm or less of average particle diameter as an active substance, wherein a melting point of a fat composition which composes an oil phase of the emulsion is 37° C. or less, and wherein the fat composition comprises 30% by weight or more of BX2 triacylglycerol wherein B represents Behenic acid and X represents fatty acid having 4 to 24 carbon atoms.

2. The agent for delaying digestion and absorption of lipid according to claim 1, wherein an amount of the BX2 triacylglycerol with respect to fat amount derived from fat-containing food ingested simultaneously with the agent is 0.5 to 20% by weight.

3. The agent for delaying digestion and absorption of lipid according to claim 1, wherein the oil-in-water emulsion is used in dried form.

4. A method for enhancing a lipase inhibitor, comprising preparing an oil-in-water emulsion having 2 μm or less of average particle diameter and administering the oil-in-water emulsion, wherein a melting point of a fat composition which composes an oil phase of the emulsion is 37° C. or less, and wherein the fat composition comprises 30% by weight or more of BX2 triacylglycerol wherein B represents Behenic acid and X represents fatty acid having 4 to 24 carbon atoms.

5. The method for enhancing a lipase inhibitor according to claim 4, wherein an amount of the BX2 triacylglycerol with respect to fat amount derived from fat-containing food ingested simultaneously with the oil-in-water emulsion is 0.5 to 20% by weight.
